(19) United States
(12) Patent Application Publication
(10) Pub. No.: US 2019/0056446 A1
(43) Pub. Date: Feb. 21, 2019

(54) METHOD AND SYSTEM FOR ASSESSMENT OF FAULT SEVERITY, RISK EXPOSURE, AND GASSING STATUS FOR LIQUID-FILLED HIGH-VOLTAGE APPARATUS

(71) Applicant: DELTA-X RESEARCH INC., Victoria (CA)

(72) Inventor: James J. DUKARM, Victoria (CA)

(21) Appl. No.: 16/059,412

(22) Filed: Aug. 9, 2018

Related U.S. Application Data
(60) Provisional application No. 62/548,062, filed on Aug. 21, 2017.

(51) Int. Cl. G01R 31/02; G01N 33/00; H01F 27/12

(52) U.S. Cl. CPC G01R 31/027; H01F 27/12; G01N 33/0063; G01N 33/004

(57) ABSTRACT

A method for assessment of fault severity, risk exposure, and gassing status for a liquid-filled high-voltage apparatus involves taking a series of samples from a liquid-filled high-voltage apparatus at intervals over a time period and subjecting those samples to gas analysis to measure and record the concentrations of selected gases. A fault energy index is computed for each of the insulating liquid samples based upon the concentrations of the selected dissolved gases for that sample. Gassing events are identified where there is a continuous production of fault gases for a time period causing an increase in the fault energy index. A computer is used to calculate a severity of each gassing event and a cumulative severity of multiple gassing events collectively, where each severity is a based on probabilities of failure provided by a reliability model comprising a random variable representing failure-related values of the fault energy index. Risk exposure is calculated by multiplying a severity value by a cost factor such as replacement cost or MVA rating. The gassing status of an apparatus is a value suitable for ranking apparatus and is determined on the basis of the severity and timing of gassing events of the apparatus.

### FIELD

The invention relates to the screening or monitoring of the condition of liquid-filled high-voltage electrical apparatus.

### BACKGROUND

Many kinds of high-voltage apparatus, such as power transformers, reactors, rectifiers, and transmission cable, are filled with a liquid for cooling and internal insulation, called “insulating liquid” in this disclosure. Liquids used for this purpose include mineral oil, polydimethylsiloxane, vegetable oils, benzene, and others.

The internal electrical insulation system of such apparatus often comprises a solid part made of cellulosic material such as kraft paper, wood, and pressboard. Alternatively the solid insulation may be made primarily of aramid polymers or other suitable materials.

When an apparatus is in good condition and working under its rated conditions of temperature, moisture, current, voltage, and magnetic flux, its liquid and solid insulating materials are chemically stable, with very slow rates of decomposition and deterioration as required for decades of service life.

Defects, severe disturbances in the electrical circuit, high temperatures in the environment, overloading, failure of cooling pumps, and many other circumstances can lead to malfunction of the apparatus (called a “fault”) with resulting exposure of the liquid and solid internal insulation to thermal and electrical stress beyond the tolerance levels of the insulating materials. Fault energy is energy dissipated in the internal insulating materials of a liquid-filled high-voltage apparatus as a result of a fault. The fault energy is sometimes sufficient to cause those materials to emit gases, called “fault gases,” that they do not emit in significant quantities during normal operating circumstances and that are not emitted in significant quantities by any other internal materials of the apparatus under any gassing circumstances. The fault gases dissolve in the liquid insulation and may also accumulate as free gas in a gas space in the apparatus or in an attachment (such as a Buchholtz relay). The concentrations of individual fault gases and their relative proportions provide evidence as to the nature and intensity of the fault process that generated them.

It is common practice to collect a sample of insulating liquid from liquid-filled high-voltage apparatus from time to time, subjecting the sample to dissolved-gas analysis (DGA) to detect and quantify the individual dissolved gases, including fault gases. An alternative form of gas analysis is to sample and analyze the free gas in a gas space of the apparatus to detect and quantify individual gases in the gas mixture found. Both DGA and free gas analysis are referred to in this disclosure as “gas analysis.” The term “sample” refers throughout this disclosure to any insulating liquid sample or any free gas sample collected from an apparatus for gas analysis.

The quantities of gases measured by a gas analysis are conventionally expressed as gas concentrations by volume in microliters per liter (μL/L) but may be expressed in other units such as moles per liter (mol/L). Gas concentrations are conventionally reduced to standard temperature and pressure conditions, such as 273.15 K and 101.325 kPa, for reporting and computational purposes.

Chromatographic analysis of free and dissolved gases in transformers has been practiced by analytical laboratories since 1968 or earlier. Automated analysis of free and dissolved gases in transformers by online monitors has been performed since the 1990's. Likewise, portable gas analyzers have been available for use in electric substations, industrial plants, and other transformer sites since the 1990's.

The mechanism of formation of the gases sampled from a liquid-filled high-voltage apparatus in either dissolved or free form is essentially the same, and therefore the interpretation of their concentrations is also the same regardless of whether the gases were free or dissolved when sampled. The primary difference between dissolved-gas analysis and the analysis of free gas is that dissolved-gas analysis begins with the extraction of the dissolved gas from the liquid, or with equilibration of the liquid with a gas space, so that the measurement of gas concentrations can be performed on the resulting free gas. For the analysis of a free gas sample, the extraction step is unnecessary, and the rest of the measurement procedure is the same as for DGA. When dissolved-gas concentrations are desired, free gas concentrations can be converted to dissolved-gas concentrations in liquid at equilibrium with the free gas by multiplication by a respective partition coefficient for each gas.

Gas analysis is a widely used and effective method for screening and monitoring liquid-filled high-voltage apparatus to provide detection, diagnosis, and assessment of problems (faults) that could lead to damage, forced outage, or failure. Conventionally the results of gas analysis interpretation for an individual apparatus include:
1. A determination of whether the apparatus appears to be functioning normally; and
2. If it seems to be not functioning normally, a statement of what fault type appears to be responsible and a rough classification (such as a numeric “condition code”) of the apparent severity of the fault.

At regular intervals, a representative sample is collected from the apparatus and analyzed for gas content. The number of gases included in the gas analysis performed by a portable gas analyzer or an online monitors may be lower than the number of gases included in the gas analysis performed by a laboratory.

The interpretation of gas analysis data requires consideration of past and present samples from the same apparatus to determine whether there is any sign of fault-related gas production, especially if it is recurrent or persistent, and if so, to calculate increments or average rates of increase. The date when the sample was collected is recorded with the sample's gas concentrations and is referred to as the sample date. When samples are collected from the same apparatus more than once in a single day, the sample time is also recorded. The sample date and sample time are used for calculating average rates of change of gas concentrations between samples.

For example, if the methane (CH₄) concentrations for samples 1 and 2 are, respectively, c₁ and c₂, and if the samples were collected t days apart, where t may have a fractional part, then the methane concentration increment is c₂−c₁, and the average rate of change (μL/L per day) of methane concentration between those samples is

$$r = \frac{{c}_{2} - {c}_{1}}{t} \qquad ( 1 )$$

Conventionally, gas analysis fault detection and severity assessment are based on comparison of gas concentrations and their increments and rates of change with respective reference limit values, as described in published DGA guides such as IEEE Std C57.104-2008 and IEC 60599-2015. In general the limit values are based on engineering considerations alone or on statistics calculated from a large database of gas analysis data.

Statistics conventionally used for gas analysis limits are the 90th and 95th and sometimes 98th percentile concentrations for each gas. The 90th percentile is commonly regarded as the upper limit for the “typical” range of gas concentration values, so that a concentration above the 90th percentile would be considered unusually high and potentially fault-related. The 95th percentile is commonly used as an “alarm” limit above which a gas concentration would be considered clearly excessive, possibly indicating a high level of deterioration. The 98th percentile, if used, is commonly interpreted as representing a very extreme gas concentration, potentially casting doubt upon the suitability of the transformer for continued service.

The IEC 60599-2015 DGA guide presents a method for deriving a purported pre-failure gas concentration limit (PFGC) and then defines an alarm gas concentration limit (AGC) as a multiple of the PFGC. The three limits for each fault gas—90th percentile, PFGC, and AGC—are used in combination to classify a gas concentration as typical (below the 90th percentile), unusually high, pre-failure, and extremely high (supposedly implying high risk of imminent failure). This scheme of classifying gas analysis results is clearly similar, if not equivalent, to the percentiles-based scheme described above.

The use of statistical survival analysis as a basis for defining limits for gas concentrations and gas sums has also been advocated. This requires the application of standard methods of reliability statistics to gas analysis data in combination with apparatus failure data to derive a survival probability curve, and from that to derive limit values corresponding to selected survival probability values such as 99%, 97%, and 95%. Depending on the gas concentrations or gas sums concerned, such limits may be applicable only in cases where a specific fault type is responsible for the fault gas production. For example, limits may be provided for methane (for partial discharge faults and thermal faults below 300 degrees C.), ethane (for thermal faults between 300 and 700 degrees C.), ethylene (for thermal faults above 700 degrees C.), and acetylene (for electrical sparking and arcing faults). Correspondingly, the decrease in survival probability (or equivalently, the increase in failure probability) associated with an increase in a fault gas concentration or fault gas sum has been proposed as a measure of severity of fault gas production.

An example of gas concentration limit values based on engineering considerations alone is the limits for acetylene (C₂H₂) provided in Table 1 of the IEEE Std C57.104-2008 transformer DGA guide. There, two microliters per liter (2 μL/L) is stated as the lower limit for Condition 2, the “greater than normal” range. Commonly in large transformer gas analysis databases it is found that the 90th percentile acetylene concentration is zero, and in many cases the 95th percentile acetylene concentration is also zero or very close to zero. To avoid the absurdity and impracticality of having zero as a gas concentration limit for acetylene, the IEEE acetylene limits were set on the basis of engineering judgment and experience, not on the basis of statistics.

The use of limit values for gas concentration rates of increase is recommended by both the IEEE and the IEC DGA guides. Limits for rates of increase, if not determined by engineering judgment, are conventionally determined by ad hoc statistical methods, such as by using the 90th percentile value of a population of gas concentration rates of change defined as average rates of change between consecutive samples.

The severity of a presumed fault, if any, conventionally may be quantified numerically as a “condition code” based upon which combinations of level and rate limits are exceeded for each gas. The IEEE Std C57.104 gas analysis condition code is conventionally treated as a fault severity indicator, a classification of relative likelihood of failure, and a classification of suitability for remaining in service, all in one number. Even in conventional gas analysis interpretive schemes that do not provide an explicit condition code (such as the method presented in IEC 60599-2015), the relative degrees of concern, presumed deterioration, and propensity to fail based on the limits provided are very similar to the meanings attributed to condition code values.

One example of a conventional scheme for deriving a condition code (1 to 4) from gas analysis data for an apparatus filled with mineral oil is as follows. For each of the gases hydrogen (H₂), methane (CH₄), ethane (C₂H₆), ethylene (C₂H₄), acetylene (C₂H₂), carbon monoxide (CO), and carbon dioxide (CO₂), provide 90th, 95th, and 98th percentile concentration limits; and 90th percentile increment and rate of increase limits. (For acetylene, supply concentration, increment, and rate limits according to engineering judgment if the percentile-based limits are unsatisfactory). Assign to each gas a “score” of 4 if its latest reported concentration is greater than its 98th percentile, 3 if greater than its 95th percentile, 2 if greater than its 90th percentile, and 1 if not greater than its 90th percentile. For each gas with a score less than 4, add 1 to the score if either the most recent increment or the most recent rate of change is greater than the respective 90th percentile. Now take the maximum of all the gas scores as the gas analysis condition code for the apparatus.

The condition code values are conventionally interpreted as failure risk classification levels, where for example a transformer whose most recent condition is 1 (no fault detected) is considered to be at low risk of imminent failure, but a transformer whose most recent condition code is 4 (high fault gas levels, possibly with high rate of increase) is considered to be deteriorated and at high risk of imminent failure. Accordingly, gas analysis condition codes are commonly used as factors in an apparatus health index used for prioritizing apparatus for maintenance or replacement.

An international metrology standard, “Evaluation of measurement data—Guide to the expression of uncertainty in measurement,” JCGM 100:2008, published by the Joint Committee for Guides in Metrology in 2008, defines measurement uncertainty as a “parameter, associated with the result of a measurement, that characterizes the dispersion of the values that could reasonably be attributed to the measurand.” That metrology standard also specifies mathematical methods of uncertainty propagation for obtaining the uncertainty of a quantity calculated from measurement quantities.

Depending on many factors such as sampling technique, sample handling between the field and the laboratory, instrument configuration and calibration, and the skill of the instrument operator, the relative measurement uncertainty of a moderate gas concentration in a sample has been found to be as low as 3% in some cases and as high as 65% or more in other cases. When comparing a gas concentration with a limit, it is essential to take the measurement uncertainty into account to judge whether the limit is exceeded with high certainty.

For calculated increments and average rates of change, it is important to determine the relative uncertainty, based on the known or assumed measurement uncertainty of the gas concentrations, to judge whether the increment and the average rate of change are statistically distinguishable from zero and whether either of them exceeds its respective limit with high certainty. The relative uncertainty u_{d} of the increment d=c₂−c₁ in equation (1) is

$${u}_{d} = u \frac{\sqrt{{c}_{2}^{2} + {c}_{1}^{2}}}{{c}_{2} - {c}_{1}} \qquad ( 2 )$$

where u is the relative measurement uncertainty of c₁ and c₂. The relative uncertainty of the average rate of change r=d/t is also u_{d}, provided that the uncertainty of t is zero.

When limit comparisons are used for interpreting gas analysis data, especially if calculated rates of change are involved, poor data quality (in particular, high measurement uncertainty) reduces sensitivity (probability of detecting a fault if there is one), reduces specificity (probability of correctly recognizing a fault-free condition), and increases the likelihood of mis-estimating fault severity.

These facts about gas analysis measurement uncertainty and its effects on fault assessment are known in the art, having been disclosed in a paper titled “Improving the reliability of transformer gas-in-oil diagnosis,” authored by M. Duval and J. Dukarm and published in the IEEE Electrical Insulation Magazine in 2005.

For an apparatus filled with mineral oil, total dissolved combustible gas (TDCG) is defined as:

TDCG=[H₂]+[CH₄]+[C₂H₆] [C₂H₄]+[CH₂H₂]+[CO] (3)

where gas names in square brackets denote the respective dissolved-gas concentrations (μL/L) from a single sample, expressed under standard temperature and pressure conditions such as 273.15 K and 101.325 kPa.

Some conventional gas analysis interpretive methods, such as the one prescribed by IEEE Std C57.104-2008, calculate TDCG for each sample and treat it as a generically representative “gas concentration” suitable for trending, fault detection, and condition code evaluation by comparison with its own statistical level and rate limits. Due to widely recognized drawbacks of TDCG for the described purpose (such as low sensitivity to high-energy fault types), alternatives to TDCG have been proposed in the scientific literature. Those alternatives are 20 typically gas sums (such as total dissolved hydrocarbon gas), weighted gas sums, or energy-weighted gas sums (where the respective weights are proportional to the amount of fault energy required to produce a standard amount of each gas).

Some energy-weighted gas sums are fault energy indexes. An energy-weighted sum of fault gas concentrations, where the relevant gases are produced principally in response to faults and principally by only one component material of the internal insulation of the apparatus, is a fault energy index. Furthermore, if E is any fault energy index and g(x) is any real-valued function which is increasing and continuous for all x>0, then g(E) is also a fault energy index. The uncertainty of a fault energy index E is determined by applying standard uncertainty-propagation methods to E, starting from the respective relative uncertainties of the gas concentrations used for computing E. Examples of fault energy indexes are defined by formulas (9), (10), and (11) in the Examples part of the Detailed Description section below. The result of applying the natural logarithm function ln(x) to any of those examples is also a fault energy index.

Note that TDCG is not a fault energy index, since carbon monoxide as a fault gas is produced principally by the cellulosic (solid) insulation, and hydrogen is produced from both the mineral oil (liquid) and cellulosic (solid) insulation and can be produced in significant quantity by non-fault-related processes. All the other gases making up TDCG are hydrocarbon gases produced almost exclusively by the mineral oil (liquid) insulation in response to faults.

When originally introduced, energy-weighted gas sums were presented as improved alternatives to TDCG, to be interpreted by means of their own respective level, increment, and rate limits analogous to those used for gas concentrations. The chief advantage of using TDCG or a weighted gas sum instead of multiple fault gas concentrations for deriving a condition code in conventional gas analysis is the reduction in complexity of fault severity assessment—only one set of limits is required, instead of the six or seven sets of limits required when individual fault gas concentrations are interpreted.

If significant gas production has occurred and is believed to be fault-related, any of several established methods (not the subject of this invention) can be applied to identify the nature of the responsible fault process and to say whether the solid insulation appears to be affected. Conventionally identified fault types are listed in the IEC 60599-2015 DGA guide. They are: corona or partial discharge (PD); low-, medium-, or high-range thermal faults(T1, T2, T3); low- or high-intensity electrical discharges (D1, D2), and thermal problems with electrical discharges (DT). Commonly used fault type identification methods are the Duval triangle or pentagon and the Rogers gas ratio method. Various proprietary fault type identification methods are also available in commercial software.

### SUMMARY

According to one aspect, there is provided a method for assessment of fault severity, risk exposure, and gassing status for a liquid-filled high-voltage apparatus. A first step involves collecting a series of samples from a liquid-filled high-voltage apparatus at intervals over a time period. A second step involves performing a gas analysis on each of the samples to determine concentrations of fault gases. A third step involves storing in a computer database a chronological history relating to each of the samples including the date of the taking of each of the samples and the gas concentration values for each of the samples from the apparatus. If the time between samples is less than one day, the sample collection time must also be recorded for each sample. A fourth step involves programming a computer to perform a series of inter-related calculations. The computer calculates a fault energy index E for each of the samples based upon the gas concentrations provided by the gas analysis. The computer then identifies, from changes in the fault energy indexes over time, E-gassing events in which there appears to be a continuous increase of E for a time period. The computer is then able to calculate a severity of each E-gassing event and a cumulative severity of all of the E-gassing events, using failure probabilities provided by a reliability model comprising a random variable representing the value of E just prior to a failure-related forced outage.

The method includes the case where several fault energy indexes, each representing a different component of the internal insulating material of the apparatus, are calculated and assessed as described above, each having its own reliability model as a basis for calculating gassing event severity and cumulative severity.

According to another aspect, there is provided a system for assessment of fault severity, risk exposure, and gassing status for a liquid-filled high-voltage apparatus in accordance with the above method. An automated sampling device or a human sampler collects a series of insulating liquid samples from a liquid-filled high-voltage apparatus at intervals over a time period. A gas analysis instrument is used for performing a dissolved-gas analysis on each of the insulating liquid samples to determine concentrations of selected gases. A computer database is provided for storing a chronological history relating to each of the insulating liquid samples, including the date and optionally the time of the collection of each of the insulating liquid samples and the gas concentration values for each of the samples collected from the liquid-filled high-voltage apparatus. A computer processor is programmed to perform a series of calculations in accordance with the method described above. The computer processor first calculates a fault energy index E for each of the insulating liquid samples based upon the gas concentrations provided by the gas analysis. The computer processor then determines, from changes in the fault energy index E over time, E-gassing events in which there is a continuous production of fault gases for a time period. The computer finally calculates a severity of each E-gassing event and a cumulative severity of all of the E-gassing events, where the severities are defined in terms of probabilities of failure provided by a reliability model comprising a random variable representing the value of E just prior to a failure-related forced outage.

Preferred embodiments of the system compute and assess several fault energy indexes, each representing a different component of the internal insulating material of the apparatus. For each fault energy index E included, the processing is as described above, each fault energy index having its own reliability model as a basis for calculating gassing event severity and cumulative severity.

### Advantages:

The advantages provided by the invention compared to systems and methods based on conventional gas analysis are:

(a) Outputs are directly useful for engineering purposes. Cumulative severity, risk exposure, and asset status are directly useful for asset management and engineering purposes. Conditional failure probabilities and economic or other risk are desirable quantities for engineering. School-type grades based on percentiles, as provided by conventional gas analysis, are less useful and harder to interpret in engineering terms.

(b) Reduce expert time and effort required for evaluating gas analysis data and reaching practical decisions about asset status and disposition.

(c) Simplicity. No reference limits are required. There are no ad hoc decision criteria. The method of the invention is based on chemical thermodynamics and apparatus reliability statistics, with direct reference to fault energy affecting each principal component of the internal insulation system. In conventional gas analysis, a large number of reference limits (typically three concentration limits and two rate limits for each of six gases) is required, none of which has a proven quantified connection with apparatus reliability.

(d) Direct empirical connection with apparatus reliability. Asset status and gassing event severity are calculated from asset failure probability as provided by reliability engineering statistics, not determined by conventional choice of percentiles or published limits. Severity and cumulative severity are conditional failure probabilities suitable for risk exposure calculations. The IEC's “probability of failure in service” method for deriving the PFGC and AGC limits cited in IEC 60599-2015 and described in CIGRE Technical Bulletin 296 (published by CIGRE in June 2006) is unfortunately not based on probability of failure as claimed in the documents cited, but instead on the probability, which can vary drastically according to sampling frequency, of finding a failure-related sample in a collection of samples.

(e) Robustness relative to gas loss. Many transformers lose gas by leakage through faulty gaskets, by occasional expulsion of headspace gas through a pressure relief valve, and in other ways. Gas loss can mask or greatly reduce the apparent severity of problems that are assessed conventionally by means of limit comparisons. Because the new method only considers periods of active gassing, when the rate of gas formation greatly exceeds the rate of gas loss, and does not depend much on the absolute magnitude of the gas concentrations, it is affected much less by gas loss than conventional gas analysis interpretation, where limit comparisons do not take gas loss into account.

(f) Superior fault sensitivity (probability of detecting faults that are present) compared to conventional gas analysis. The new method was found by Duke Energy to detect thermal problems in power transformers, some quite severe, that were undetected by conventional gas analysis. When applied to a transformer failure case used as an example by Qualitrol Corp. for their online monitoring gas analysis software product, the invention detects the fault as of a date one week earlier than the Qualitrol software does. The customary recommendation in conventional gas analysis to wait for some gas concentration to exceed its 90th percentile limit before investigating or taking mitigative steps makes conventional gas analysis less sensitive to potentially dangerous incipient faults.

(g) Superior selectivity (probability of finding no fault when there is none to find) compared to conventional gas analysis. When the new method was tested by Duke Energy, a large number of transformers that were erroneously classified as abnormal by conventional gas analysis because of static gas levels exceeding a concentration limit were classified as acceptable (status 1 or 2) by the new method. Reliability statistics show clearly that, for a transformer that does not continue to generate fault gas, high concentrations of residual fault gases are not indicative of increased failure rate per unit of concentration or per unit of energy index.

(h) Generality. The invention is applicable to gas analysis at all sampling rates including online monitoring. With due consideration of signal processing issues (low vs. high sampling rate, high vs. low measurement uncertainty) relevant to detecting gassing event endpoints, which is not part of this invention, the interpretive method is applied identically to both kinds of gas analysis data. Because of its reliance on 90th percentile thresholds, conventional gas analysis applied to online monitor data is insensitive to incipient problems and inclined to misclassify non-gassing apparatus as deteriorated. Because the statistical rate of change limits employed by conventional gas analysis are based on sampling intervals of several weeks or months, those limits when applied to online monitor data (with sampling rate of a few hours and with “noisy” short-term variation) tend to produce false alarms or to overlook long-term fault gas production at rates falling short of the limits.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a block diagram of the system of the invention.

FIG. 2 is a bar chart showing the standard enthalpies of formation of four hydrocarbon gases from n-octane.

FIG. 3 is a bar chart showing the standard enthalpies of formation of two carbon oxide gases from glucose, a model of a cellulose monomer.

FIG. 4 is a probability density chart for the lognormal random variable of failure-related values of NEI-HC.

FIG. 5 is a cumulative density (failure probability) chart for the lognormal random variable of failure-related values of NEI-HC.

FIG. 6 is a probability density chart for the lognormal random variable of failure-related values of NEI-T.

FIG. 7 is a cumulative density (failure probability) chart for the lognormal random variable of failure-related values of NEI-T.

FIG. 8 is a probability density chart for the lognormal random variable of failure-related values of NEI-CO.

FIG. 9 is a cumulative density (failure probability) chart for the lognormal random variable of failure-related values of NEI-CO.

FIG. 10 is a table of fault gas concentration data for the transformer in the example. Each row of the table corresponds to one sample.

FIG. 11 is a hydrocarbon gas NEI (NEI-HC) time series chart, with NEI-HC gsssing events marked with dashed rectangles, for the transformer in the example.

FIG. 12 is a table in which each row describes an NEI-HC gassing event shown in FIG. 11.

FIG. 13 is a carbon oxide gas NEI (NEI-CO) time series chart, with one NEI-CO gsssing event marked with a dashed rectangle and another NEI-CO gassing event marked with a dotted rectangle, for the transformer in the example.

FIG. 14 is a table in which each row describes an NEI-CO gassing event shown in FIG. 13.

### DETAILED DESCRIPTION

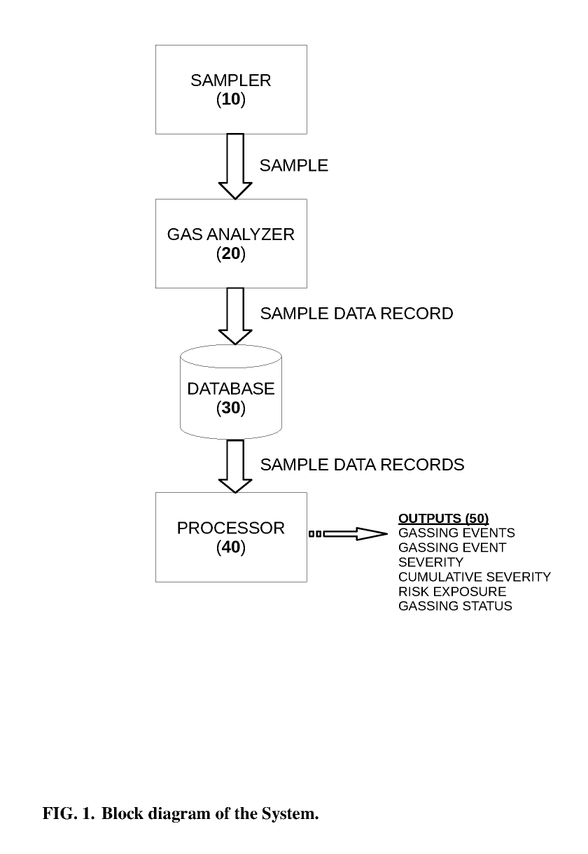

A method and system for assessment of fault severity, risk exposure, and gassing status for liquid-filled high-voltage apparatus will now be described with reference to FIG. 1 through FIG. 14.

The method of this invention does not assume or depend on any particular units or standard conditions used for expressing gas concentrations.

### Method:

The method requires that at least one fault energy index, relating to one component material of the internal insulation of the apparatus, be used.

Preferred embodiments of the method use one fault energy index for each insulation component material of the apparatus—for example, one for the liquid insulation and another one for the cellulosic (paper and wood and pressboard) insulation in a transformer.

Let A be a liquid-filled high-voltage apparatus subjected to sampling and gas analysis from time to time. The method requires that gas concentration measurement values obtained by gas analysis of a sample be recorded, along with information about when the sample was collected, in a persistent data structure, referred to as a “sample data record,” which in turn is recorded in a persistent data store, referred to as a “database.”

In preferred embodiments of the method, the database contains multiple sample data records for each of many apparatuses.

For each fault energy index E used for apparatus A, an observation of E is defined to be a value for E calculated using gas concentration measurement values in a sample data record for A. Thus, each observation of E is associated with exactly one sample data record.

An E-gassing event of a specified apparatus A is defined to be a time interval in which there is production of fault gas by the apparatus A leading to a net increase in the fault energy index E spanning the time between the initial date t₁ and the final date t₂ of the time interval. Correspondingly there are an initial value x₁ and a final value x₂ of E during that time interval.

An observation of E is defined to be failure-related if (a) it is associated with a sample collected from A within the time span of an E-gassing event, and (b) that sample is the last one collected from A within one routine sampling interval before A experienced a forced outage due to failure or impending failure of A.

The method of this invention requires, for a specified kind of appratus and for each fault energy index E used in connection with that kind of apparatus, a means of computing failure probability F_{E}(x) as an increasing continuous function of values x of E, where F_{E}(x) denotes the proportion of a population of that kind of apparatus that is expected to fail with E less than or equal to the value x. Such a means can always be understood mathematically as defining F_{E}(x) as the cumulative distribution function for a random variable X_{E} such that F_{E}(x)=Pr(X_{E}≤x). The random variable X_{E} is thus a reliability model for failure-related observations of E.

According to the method of the invention, the severity of an E-gassing event in which E increases from x₁ to x₂ is defined to be the conditional probability

sev_{E}(x₁, x₂)=Pr(x₁<X_{E}≤x₂|X_{E}>x₁) (4)

Since F_{E} is the cumulative distribution function for X_{E}, it follows that the severity (4) of a gassing event in which E increases from x₁ to x₂ can be calculated from F_{E} thus:

$${sev}_{E} ( {x}_{1} , {x}_{2} ) = \frac{{F}_{E} ( {x}_{2} ) - {F}_{E} ( {x}_{1} )}{1 - {F}_{E} ( {x}_{1} )} \qquad ( 5 )$$

Let G₁, G₂, . . . , Gₙ be a sequence of E-gassing events for apparatus A, where for each i between 1 and n the initial and final values of Eᵢ are respectively aᵢ and bᵢ, and where none of the events overlaps in time with any of the other events, and where G₁ is the earliest event. Although aᵢ<bᵢ for all i, it is possible that due to gas loss from the apparatus A, bᵢ>aᵢ₊₁ for some values of i. That is, the E value ranges of some of the events may overlap if A is not gas-tight.

According to the method of the invention, the cumulative severity of a sequence G₁, G₂, . . . , Gₙ of E-gassing events for apparatus A as described above is defined to be the conditional probability

csev_{E}(a₁, a₂, . . . , aₙ; b₁, b₂, . . . , bₙ)=Pr(a₁<X_{E}≤b|X_{E}>a₁) (6)

where b is:

$$b = {a}_{1} + {\sum }_{1 \leq i \leq n}^{ } ( {b}_{i} - {a}_{i} ) \qquad ( 7 )$$

It follows that the cumulative severity of a sequence G₁, G₂, . . . , Gₙ of E-gassing events for apparatus A as described above can be calculated thus:

csev_{E}(a₁, a₂, . . . , aₙ; b₁, b₂, . . . , bₙ)=sev_{E}(a₁, b) (8)

where b is defined as in (7) above.

Let c be any failure cost factor (such as estimated replacement cost) for apparatus A. According to the method of the invention, the risk exposure due to an E-gassing event for A with initial value E=x₁ and final value E=x₂ is defined to be the product c·sev_{E}(x₁, x₂).

Let c be any failure cost factor (such as estimated replacement cost) for apparatus A. According to the method of the invention, the cumulative risk exposure due to a sequence of E-gassing events for A with cumulative severity s is defined to be the product c·s. Note that risk exposure is not an indication of the risk of imminent failure or of increased failure rate.

The method of the invention defines the gassing status code of an apparatus A to be a code number assigned to A on the basis of the pattern and severity of the gassing events of A. Let every E-gassing event of an apparatus A, for every fault energy index E used for assessing A, be called a “gassing event” of A. The intention of the gassing status code is to provide a numerical ranking for apparatus with respect to the apparent degree of need for surveillance, maintenance, or mitigative action, in the style of the condition code defined in IEEE Std C57.104-2008. A gassing status code value of 0 denotes “no data available”; 1 denotes “no significant gassing ever”; 2 denotes “no recent significant gassing event”; and 3 denotes “recent significant gassing.” Optionally status value 4 can be defined as “recent extreme gassing.” The method does not specify how to define the significance of a gassing event. It could be based, for example, on severity or on risk exposure.

A preferred embodiment of the method defines the gassing status of an apparatus A as follows.
1. No significant gassing event ever.
2. There was at least one significant gassing event, but none recently (where the preferred meaning of “recently” for this purpose is “within one routine sampling interval”).
3. There is a recent gassing event of low to moderate severity (severity less than a predefined limit such as 2
4. There is a recent gassing event of high severity (severity equal to or exceeding a predefined limit as above).

### System:

Because of the large volume of data that must be interpreted to assess the results of periodic gas analysis testing of a fleet of liquid-filled high-voltage apparatus in an electric utility or an industrial plant, for example, it is necessary to have an organized system to acquire and organize the data, perform the assessment according to the method, and generate summary results for review by experts such as maintenance engineers and asset managers.

Referring to FIG. 1, the system used to implement the method includes a means of sampling apparatus 10, a gas analyzer 20 to perform the gas analysis on each sample collected, a database 30 for recording the analysis data, and a computer processor 40 programmed to calculate outputs 50, comprising the energy indexes, tabulated gassing events, gassing event severities and cumulative severities, and E-status of the apparatus for each fault energy index E employed. Based on those calculations, the computer can provide notifications and summary and detail results to the expert users.

### Working Example—Fault Energy Indexes

For the case of power transformers filled with mineral oil, three fault energy indexes are useful:

The hydrocarbon gas normalized energy intensity (NEI-HC) is defined as

$$NEI \text{-} HC = \frac{77.7 [ {CH}_{4} ] + 93.5 [ {C}_{2} {H}_{6} ] + 104.1 [ {C}_{2} {H}_{4} ] + 278.3 [ {C}_{2} {H}_{2} ]}{22400} \qquad ( 9 )$$

The Duval triangle gas normalized energy intensity (NEI-T) is defined as

$$NEI \text{-} T = \frac{77.7 [ {CH}_{4} ] + 104.1 [ {C}_{2} {H}_{4} ] + 278.3 [ {C}_{2} {H}_{2} ]}{22400} \qquad ( 10 )$$

The hydrocarbon gas normalized energy intensities for mineral oil defined in formulas (9) and (10) were introduced in a paper by F. Jakob and J. Dukarm titled “Thermodynamic estimation of transformer fault severity” and published in IEEE Transactions on Power Delivery in 2015.

The carbon oxide gas normalized energy intensity (NEI-CO) is defined as

$$NEI \text{-} CO = \frac{101.4 [ CO ] + 30.2 [ {CO}_{2} ]}{22400} \qquad ( 11 )$$

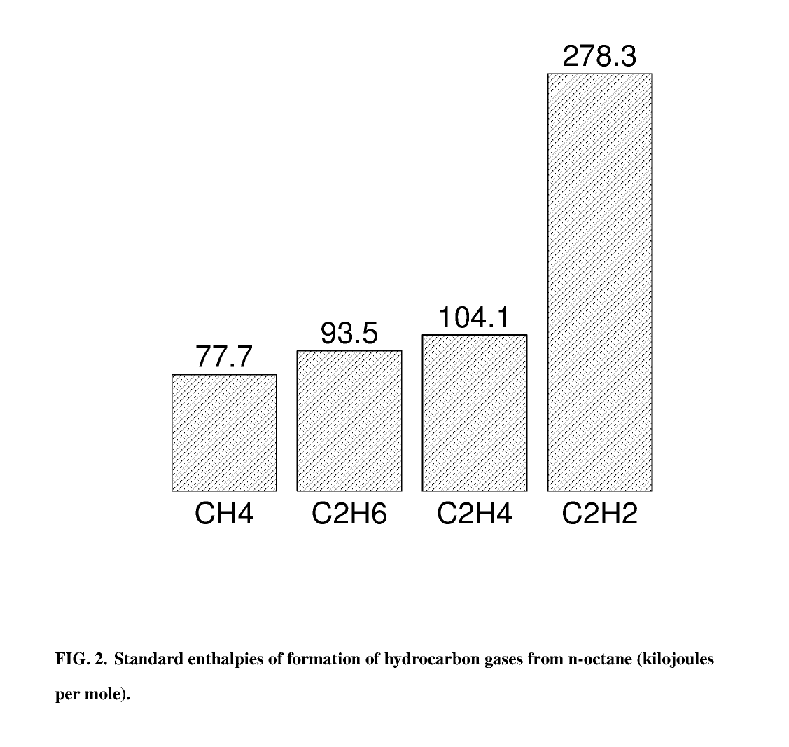

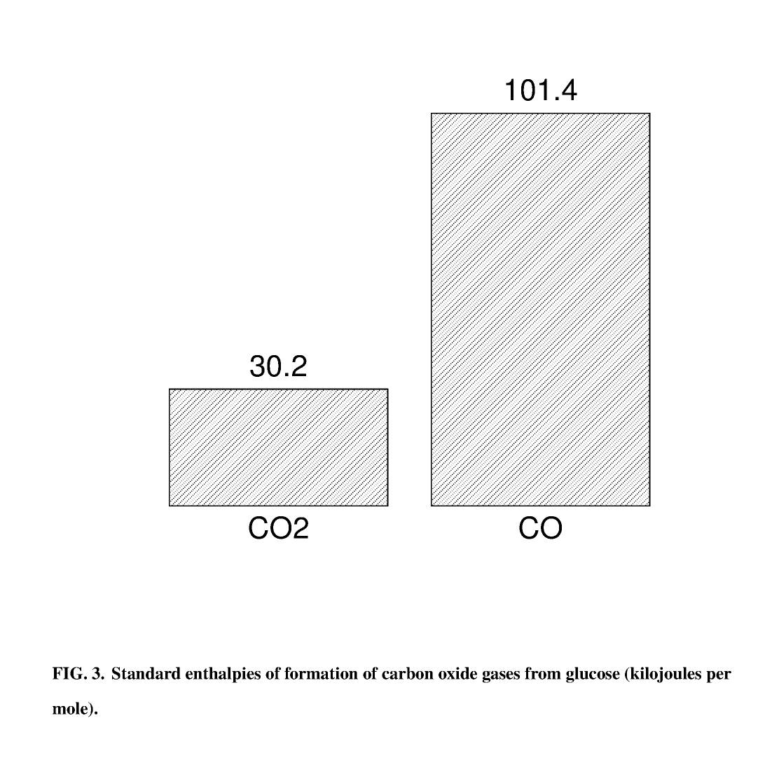

In each of the three formulas above, the bracketed gas names denote dissolved-gas concentrations (μL/L) in mineral oil, measured in the same sample and expressed at standard temperature and pressure (for example, 273.15 K and 101.325 kPa). (For this example, concentrations in free gas would need to be converted to corresponding dissolved-gas concentrations by multiplying them by the respective partition coefficients.) The numeric coefficients of the gas concentrations in the formulas are the respective standard enthalpies of formation (kJ/mol), from n-octane (C₈H₁₆, a model for a typical mineral oil molecule) for the hydrocarbon gases (see FIG. 2), and from glucose (C₆H₁₂O₆, a model for the monomer of cellulose) for the carbon oxide gases (see FIG. 3). The denominator of 22400 in each case is a units conversion factor converting from (kJ/mol)·(μL/L) to kJ/kL or, equivalently, kJ·m⁻³.

In situations where all of the gas concentrations required for both NEI-HC and NEI-CO are provided, NEI-HC is used for assessment of faults affecting the insulating oil, and NEI-CO is used for the assessment of faults affecting the solid (cellulosic) insulation.

NEI-T is used instead of NEI-HC for transformers that are suspected of ethane “stray gassing,” i.e., production of excessive amounts of ethane gas under moderate operating temperatures where no abnormality is suspected. In those cases, NEI-T is used for assessment of faults affecting the insulating oil, and NEI-CO is used for the assessment of faults affecting the solid (cellulosic) insulation.

NEI-T is also used instead of NEI-HC when the source of gas analysis data is an online gas monitor that measures the concentrations of methane, ethylene, and acetylene but not ethane.

NEI-CO can be used only when the concentrations of both carbon monoxide and carbon dioxide are being measured.

### Working Example—Reliability Model for a Fault Energy Index

A data set was compiled from gas analysis and transformer failure data supplied by two large USA electric utilities, comprising 7151 sample data records—one for each of 7151 transformers—of the form (x, tₓ), where x is the last observed in-service value of NEI-HC (defined above in formula (9), and tₓ=1 if (a) the respective transformer experienced a failure-related forced outage within one year of the date of the sample and (b) the sample was part of an NEI-HC gassing event. Otherwise, tₓ=0. Of the 7151 sample records, 101 were terminal, i.e., had tₓ=1.

A standard statistical procedure called maximum likelihood estimation (MLE) was used to fit various random variable types (including exponential, Weibull, and lognormal) to the data to estimate the type and parameters for the best-fitting probability models for the failure-related values of NEI-HC. The MLE procedure takes into account both the terminal (tₓ=1) and the nonterminal (tₓ=0) observed values to obtain the best fit of a specified type of random variable to the data. For NEI-HC the best fitting type of random variable for the data was lognormal.

For the fault energy indexes NEI-T (formula (10)) and NEI-CO (formula (11)), respective data sets were compiled as described for NEI-HC above. For both NEI-T and NEI-CO, MLE showed that the best fitting type of random variable for the data was lognormal.

If X is a lognormal random variable, then ln(X) is a normal random variable. Conventionally the parameters μ (mean) and σ (standard deviation) of that associated normal random variable are used as the parameters to describe the lognormal random variable. The probability density function for a lognormal random variable X with parameters μ and σ in that sense is

$${f}_{X} ( x ) = \frac{1}{σ x} φ ( \frac{ln ( x ) - μ}{σ} ) \qquad ( 12 )$$

where x >0 and φ is the density function of the standard normal random variable. The cumulative distribution function (also called the failure probability function or reliability function in the context of reliability statistics) for a lognormal random variable X with parameters μ and σ is

$${F}_{X} ( x ) = Pr ( X \leq x ) = {\int }_{0}^{x} {f}_{X} ( t ) dt = Φ ( \frac{ln ( x ) - μ}{σ} ) \qquad ( 13 )$$

where x>0 and Φ is the cumulative distribution function of the standard normal random variable.

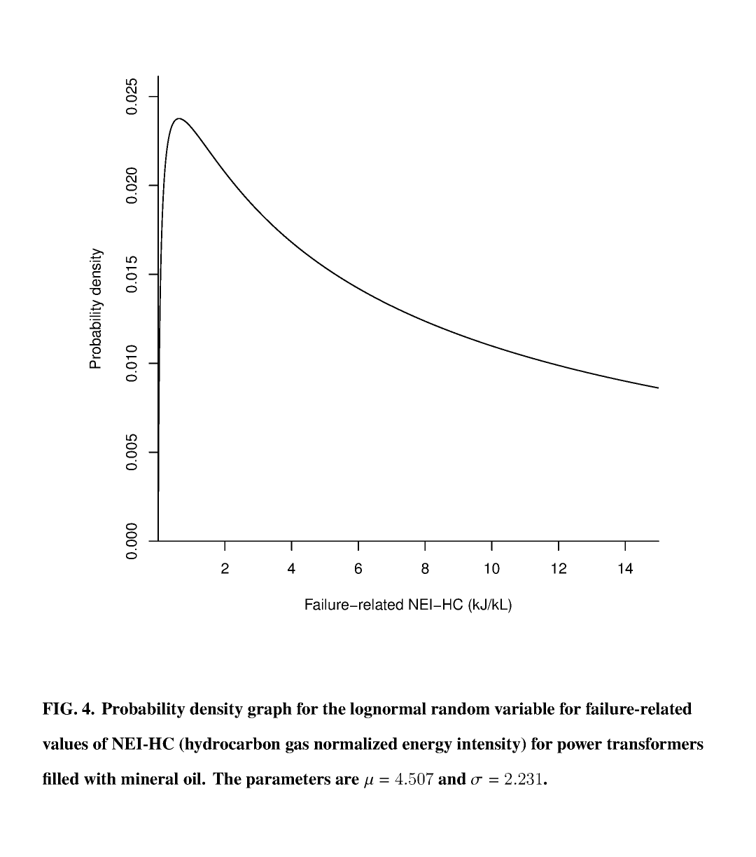

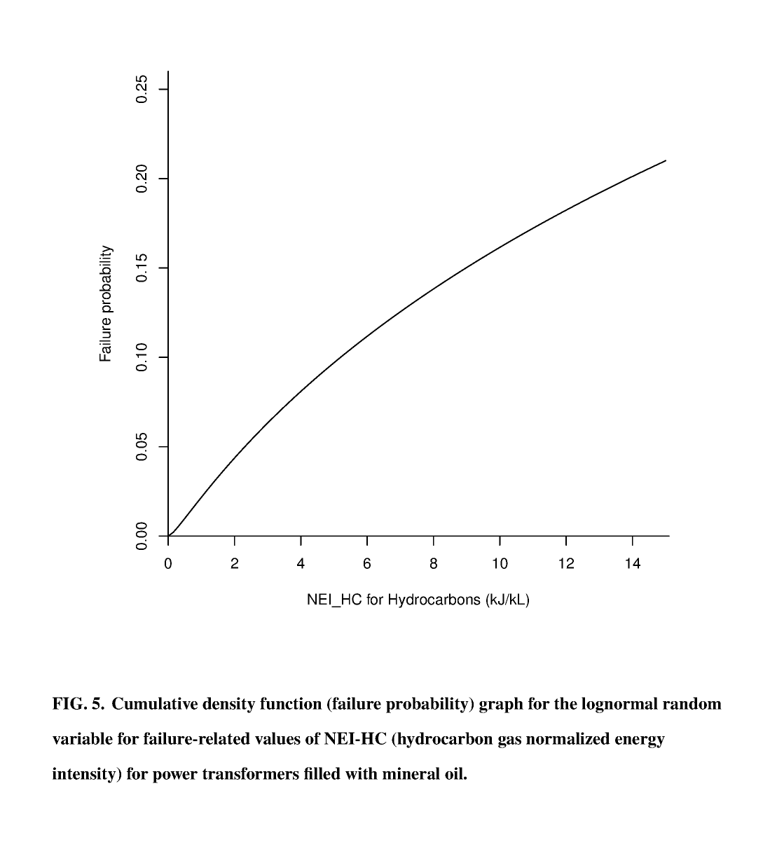

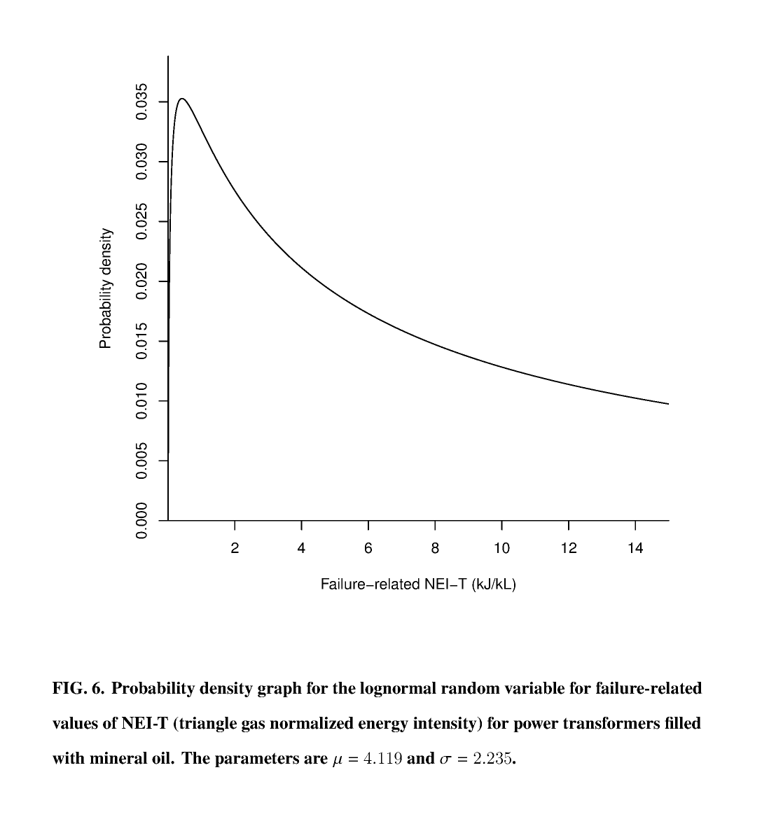

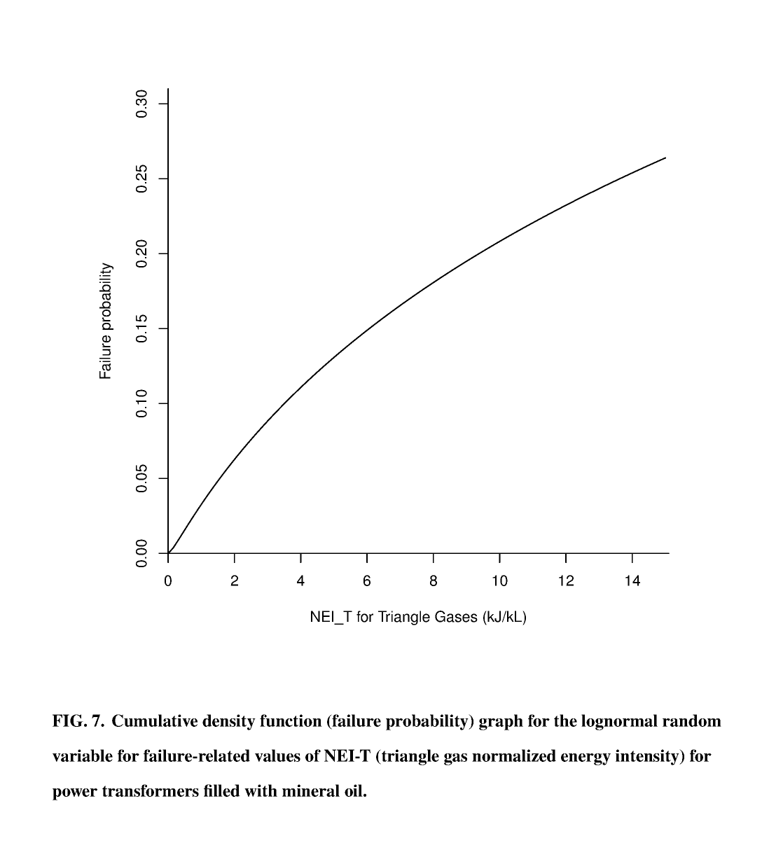

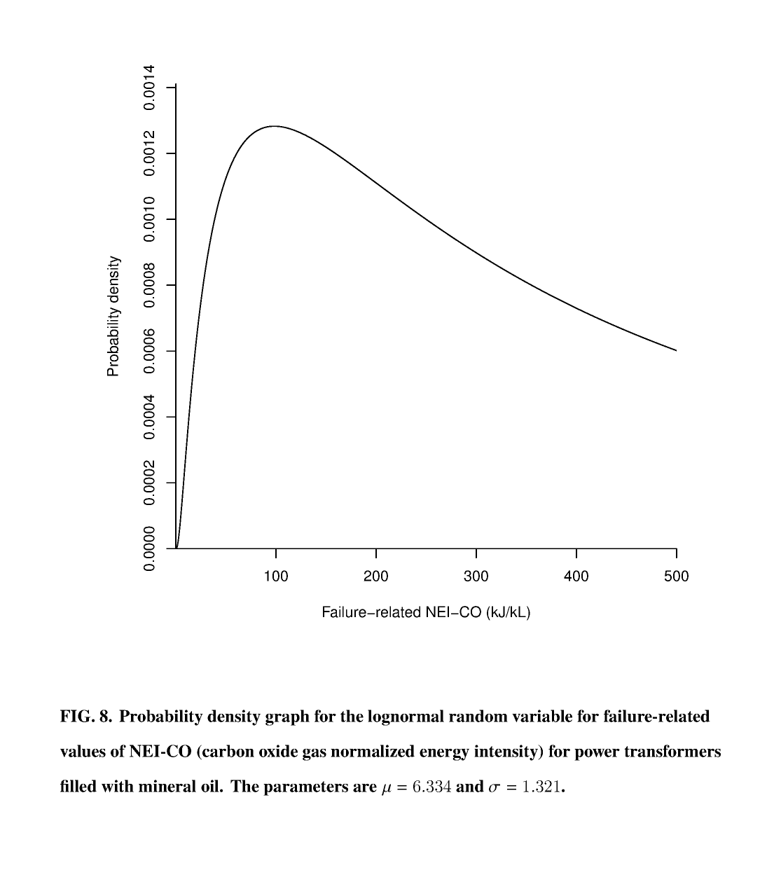

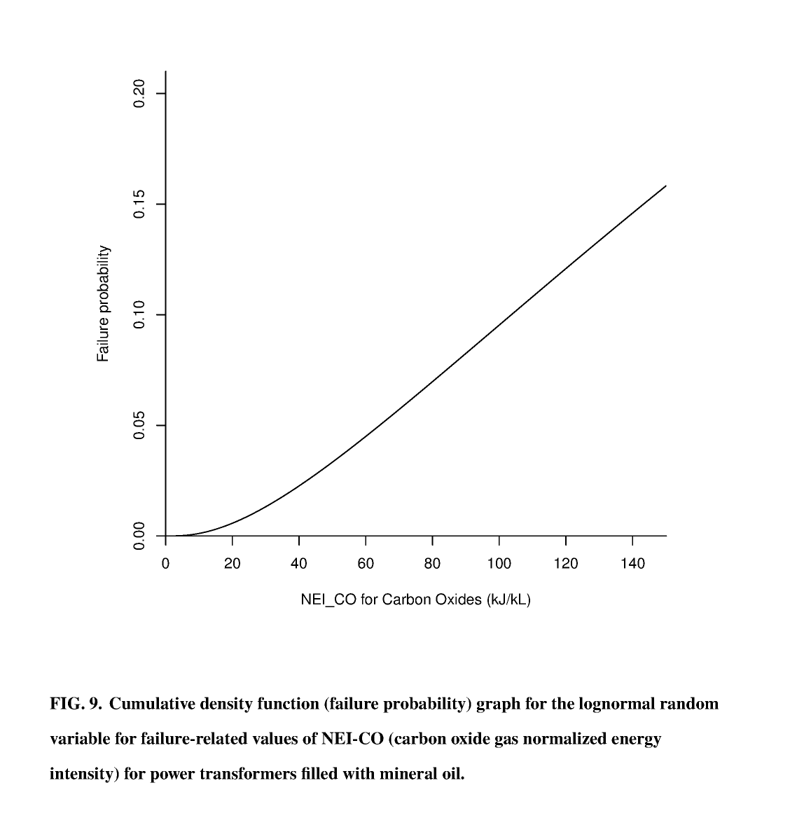

The parameters of the fitted lognormal random variables found by MLE were μ=4.507 and σ=2.231 for NEI-HC; μ=4.119 and σ=2.235 for NEI-T; and μ=6.334 and σ=1.321 for NEI-CO. The corresponding probability density graphs are shown in FIG. 4 for NEI-HC, FIG. 6 for NEI-T, and FIG. 8 NEI-CO. The corresponding failure probability graphs are shown in FIG. 5 for NEI-HC, FIG. 7 for NEI-T, and FIG. 9 for NEI-CO.

### Working Example—Assessment of a Power Transformer

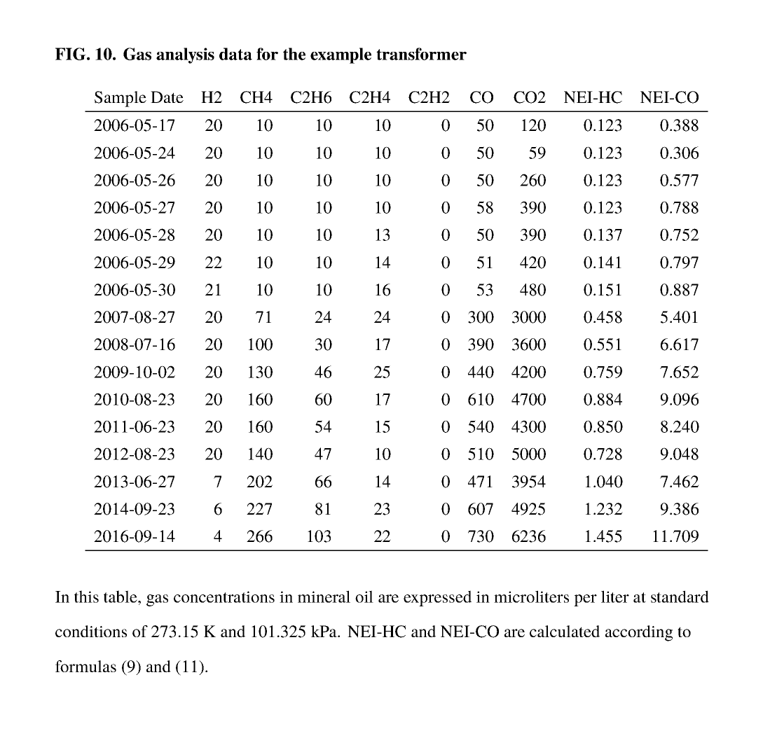

FIG. 10 is a table showing the gas analysis data, one row per oil sample, for a 140 MVA mineral-oil-filled power transformer. For this transformer the NEI-HC cumulative severity is 3.39% based on two NEI-HC gassing events. The NEI-CO cumulative severity is 0.23% based on two NEI-CO gassing events. The gassing status of the transformer is 3 because there is at least one recent gassing event. In all the gassing events noted, the apparent fault type is T1 (overheating below 300 degrees Celsius). The risk exposure based on NEI-HC cumulative severity and the MVA rating is (0.0339)(140)=4.67 MVA. Risk exposure based on NEI-HC cumulative severity and an assumed $5 million cost of failure is (0.0339)(5000000)=$169,500—an amount sufficient to warrant an investigation and possible mitigative action.

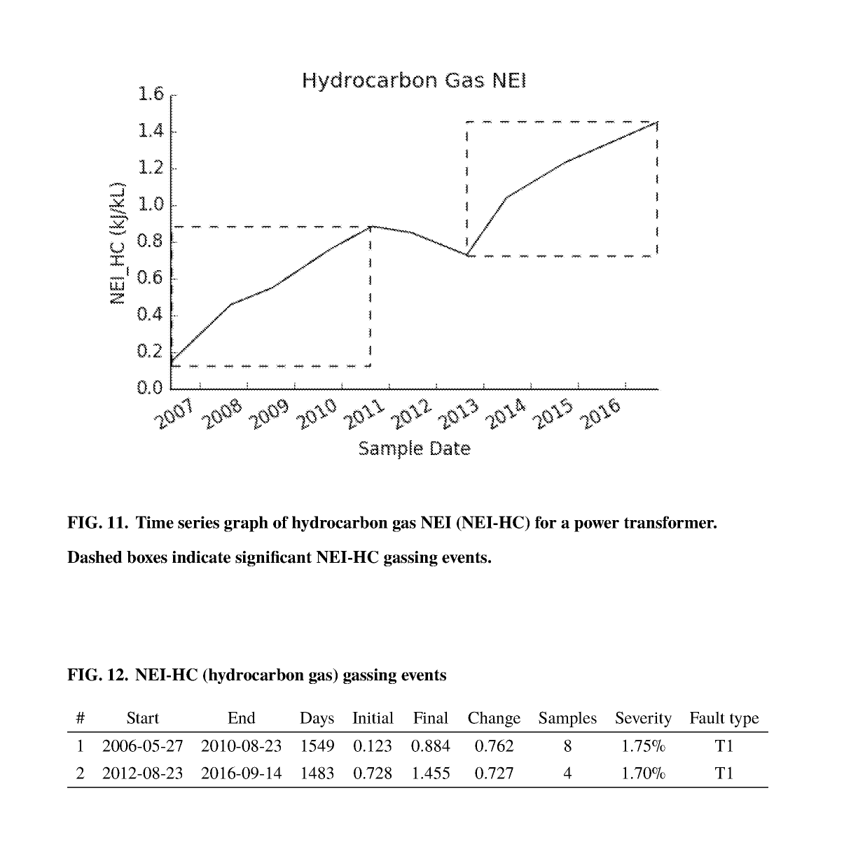

FIG. 11 is a time series graph showing NEI-HC vs. time for the transformer. The dashed boxes superimposed on the graph mark NEI-HC gassing events. FIG. 12 is a table, each row of which describes an event indicated in FIG. 11.

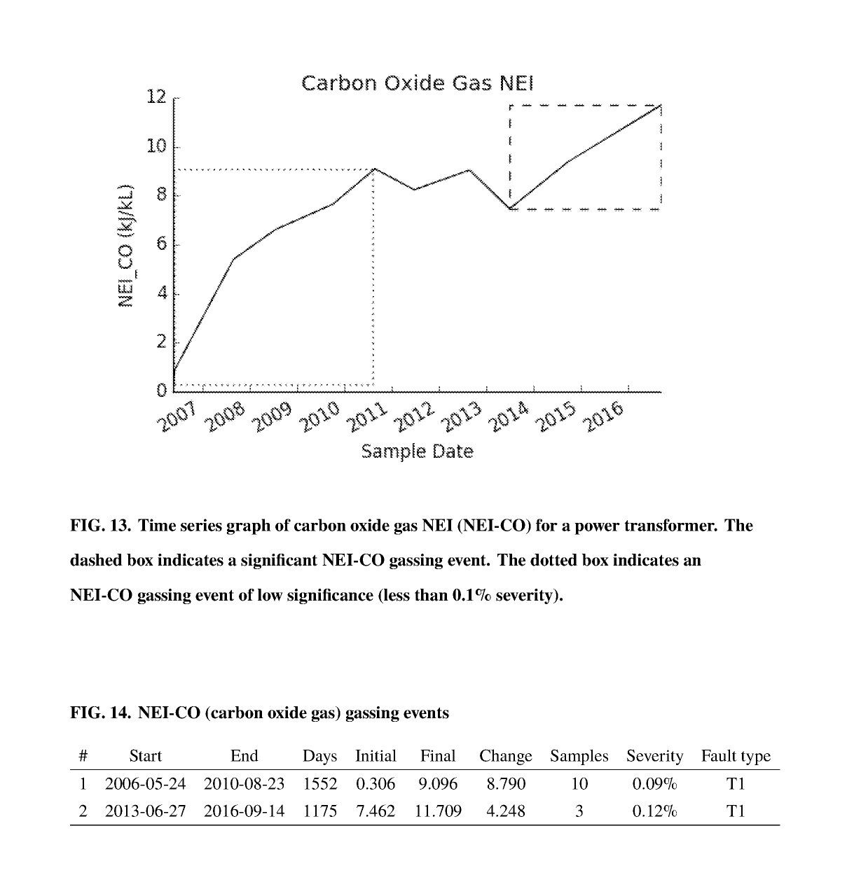

Similarly, FIG. 13 is a time series graph showing NEI-CO vs. time for the same transformer. A dashed box indicates an NEI-CO gassing event, and a dotted box indicates another NEI-CO gassing event of marginal significance (with severity less than 0.1 percent). FIG. 14 is a table, each row of which describes an event indicated in FIG. 13.

What is claimed is:

1. A Method for Assessment of Fault Severity, Risk Exposure, and Gassing Status for a Liquid-Filled High-Voltage Apparatus, comprising:
taking a series of samples from a liquid-filled high-voltage apparatus at intervals over a time period;
performing a gas analysis on each of the samples to measure concentrations of selected gases;
storing in electronic form (called “the database”) a data record for each sample containing the gas concentration measurement values pertaining to that sample as well as information (sufficient for calculating time intervals between samples) as to when the sample was collected.
programming a computer:
to calculate a fault energy index value for each of any number of selected sample data records in the database based upon the gas concentrations in the data record that are required for the calculation;
to search the sample data records pertaining to a selected apparatus in the database and tabulate the initial and final dates and initial and final fault energy index values of time intervals (“gassing events”) in which there is production of fault gas by the apparatus leading to a net increase in the fault energy index spanning the time period between the initial date and the final date;
to calculate a severity of a gassing event proportional to a conditional probability of failure derived from a reliability model comprising a random variable representing failure-related values of the fault energy index;
to calculate a gassing status code for the apparatus based on the time of occurrence and the severity of gassing events for that apparatus.

2. The method of claim 1, wherein the samples are representative insulating liquid samples taken from the apparatus and the gas concentrations are dissolved-gas concentrations.

3. The method of claim 1, wherein the samples are gas samples taken from a gas space of the apparatus and the gas concentrations measured for each sample are concentrations of selected gases in the gas space.

4. The method of claim 3, wherein the gas concentrations in the gas space are converted to dissolved-gas concentrations in the insulating liquid that would be expected when the gas concentrations in the gas space and the liquid are in equilibrium.

5. The method of claim 1, wherein for the selected apparatus a cumulative severity of selected gassing events is calculated, proportional to a conditional probability of failure derived from a reliability model comprising a random variable representing failure-related values of the fault energy index.

6. The method of claim 1, wherein the severity of an individual gassing event is multiplied by a predetermined cost consequence of a failure of the liquid-filled high-voltage apparatus, to calculate a risk exposure value.

7. The method of claim 5, wherein the cumulative severity of multiple gassing events is multiplied by a predetermined cost consequence of a failure of the liquid-filled high-voltage apparatus, to calculate a risk exposure value.

8. The method of claim 1, wherein the selected apparatus is assigned a gassing status code, suitable for ranking apparatus and determined on the basis of the severity and order of occurrence of selected gassing events of that apparatus.

9. The method of claim 1, wherein the computer is programmed to raise an alert if acetylene concentration increases during a time period spanned by multiple samples of an apparatus.

10. The method of claim 1, wherein the liquid-filled high-voltage apparatus is a mineral oil filled power transformer and the energy index is based on methane, ethylene, and acetylene concentrations.

11. The method of claim 10, wherein the fault energy index is also based on ethane concentration.

12. The method of claim 1, wherein the liquid-filled high-voltage apparatus is a mineral oil filled power transformer and the energy index is based on the carbon monoxide concentration.

13. The method of claim 12, wherein the fault energy index is also based on carbon dioxide concentration.

14. The method of claim 1, wherein multiple fault energy indexes are calculated and assessed.

15. A System for Assessment of Fault Severity, Gassing Status, and Risk Exposure for a Liquid-Filled High-Voltage Apparatus, comprising:
a sampler for taking a series of samples from a liquid-filled high-voltage apparatus at intervals over a time period;
a gas analyzer for measuring the concentrations of selected gases in samples;
a computer database for storing sample data records comprising the gas concentration measurement values for a sample as well as information (sufficient for calculating time intervals between samples) as to when the sample was collected;
a computer processor programmed:
to calculate a fault energy index value for each of any number of selected samples in the database based upon the gas concentrations that are required for the calculation;
to search the sample data pertaining to a selected apparatus in the database and tabulate the initial and final dates and initial and final fault energy index values of time intervals (“gassing events”) in which there is production of fault gas by the apparatus leading to a net increase in the fault energy index spanning the time period between the initial date and the final date;
to calculate a severity of a gassing event proportional to a conditional probability of failure derived from a reliability model comprising a random variable representing failure-related values of the fault energy index.

16. The system of claim 14, wherein multiple fault energy indexes are calculated and assessed.
